(19) United States
(12) Patent Application Publication
(10) Pub. No.: US 2024/0218347 A1
(43) Pub. Date: Jul. 4, 2024

(54) Polymer of Protein-Conjugated Polysaccharide, Preparation Method Therefor, Enzyme-Stabilizing Composition Comprising Same, and Method for Detecting Target Nucleic Acid Molecule by Using Same

(71) Applicant: SML GENETREE CO., LTD., Seoul (KR)

(72) Inventors: Yo Han Jeong, Seoul (KR); Jeong Ju Lee, Seoul (KR); So Hyeon Park, Seoul (KR); Kyung Ah Hwang, Seoul (KR); Ji Hoon Ahn, Seoul (KR)

(73) Assignee: SML GENETREE CO., LTD., Seoul (KR)

(21) Appl. No.: 17/924,446

(22) PCT Filed: Oct. 7, 2022
(86) PCT No.: PCT/KR2022/015160

§ 371 Date: Nov. 10, 2022

(30) Foreign Application Priority Data
Dec. 23, 2021 (KR) 10-2021-0186407

(51) Int. Cl. C12N 9/96; C07K 14/76; C08B 15/02; C08B 37/00; C08B 37/02; C12Q 1/6806; C12Q 1/6844

(52) U.S. Cl. CPC C12N 9/96; C07K 14/76; C08B 15/02; C08B 37/0018; C08B 37/0021; C12Q 1/6806; C12Q 1/6844

(57) ABSTRACT

The present disclosure relates to a polymer in which bovine serum albumin (BSA) is conjugated to a polysaccharide, a method of preparing the same, an enzyme-stabilizing composition including the same, and a method of amplifying or detecting a target nucleic acid molecule in a biological sample by using the same. The polymer is used to prevent the hydrolysis, oxidation, or reduction of an enzyme, such as a nucleic acid polymerase, in a composition, thereby maintaining the stability of the composition. Even when stored at room temperature and high temperature, a composition including the polymer can stably maintain enzymatic activity equivalent to that of a composition kept frozen. Accordingly, the polymer of the present disclosure and a composition including the same efficiently detect a target nucleic acid molecule associated with the onset of infectious diseases and/or cancer, and thus can be usefully applied to the diagnosis of diseases.

### CROSS-REFERENCE TO RELATED APPLICATION

This application is based on and claims priority under 35 U.S.C. § 119 to Korean Patent Application No. 10-2021-0186407 filed on Dec. 23, 2021, in the Korean Intellectual Property Office, the disclosure of which is incorporated by reference herein in its entirety.

### TECHNICAL FIELD

The present disclosure relates to a polymer, and more particularly, to a polymer in which a protein is conjugated to a polysaccharide, a method of preparing the same, an enzyme-stabilizing composition comprising the polymer, and a method of detecting a target nucleic acid by using the polymer.

### BACKGROUND ART

The COVID-19 outbreak in China in 2019 caused a global pandemic. Accordingly, PCR test, which can rapidly diagnose whether to be infected with COVID-19 or not by using a polymerase chain reaction (PCR), is attracting attention. PCR is a molecular biological technique for replicating and amplifying a target portion of deoxyribonucleic acid (DNA) or ribonucleic acid (RNA).

A specific fragment of a target nucleic acid molecule to be analyzed may be selectively amplified through PCR. PCR requires a very short time, a simple analysis process, and can be performed with a fully automatic device to efficiently amplify a specific fragment. Therefore, PCR is widely used in various fields such as molecular biology, medicine, crime, biological classification, and diagnosis.

To perform PCR, a composition including a template DNA to be replicated as a target nucleic acid molecule, primers to designate the points of initiation of DNA replication, a DNA polymerase, a probe, dNTP, and the like is used. Taq polymerase, which is mainly used in the PCR process, can maintain activity at 95° C. for about 1 hour, but loses the activity thereof due to reduced stability when stored at room temperature. To maintain activity, Taq polymerase is stored and transported in a frozen state before use. Accordingly, storage costs and time are required before PCR.

Various polymer materials and additives are added to preserve the activity of Taq polymerase, which is reduced when stored for a long period of time at room temperature. It is known that these additives maintain the activity of Taq polymerase at room temperature, thus enabling long-term storage. However, it is known that, when stored in a freeze-dried form, the activity of Taq polymerase decreases during drying and rehydration. In the case of bovine serum albumin (BSA), which is mainly added to freeze-drying, BSA does not have a significant effect on PCR, but is known to inhibit the reverse transcription (RT) process. Therefore, there is a need for a material and method in which the activity of a nucleic acid polymerase is not reduced even when stored and kept at room temperature.

### DETAILED DESCRIPTION OF THE DISCLOSURE

### Technical Problem

It is an object of the present disclosure to provide a polymer in which enzyme activity can be stably maintained even when stored at room temperature for a long period of time, and a protein is conjugated to a polysaccharide, a method of preparing the same, a polymerase chain reaction-stabilizing composition, and a method of detecting a target nucleic acid.

### Technical Solution

According to one aspect, provided is a polymer in which bovine serum albumin (BSA) is conjugated to a polysaccharide.

In one embodiment, provided is a polysaccharide-serum albumin complex including a polysaccharide and serum albumin conjugated to the polysaccharide.

The term “albumin” as used herein refers to one of the proteins constituting the basic material of cells, which is abundant in blood and produced in the liver. Albumin has the lowest molecular weight among simple proteins that exist in nature. Serum albumin in blood has a function of maintaining and restoring plasma volume, and thus prevents shock due to excessive bleeding and is used in surgery, burn treatment, and the like. It is also known that serum albumin has an oxygen transport ability similar to that of hemoglobin. Albumin may include, without limitation, human serum albumin, egg white albumin, and bovine serum albumin.

The polysaccharide may be selected from the group consisting of cellulose, hydroxypropyl cellulose, hydroxypropyl methylcellulose, glycosaminoglycan, pullulan, alginic acid, carrageenan, aribinogalactan, hemicellulose, dextran, chitosan, glycol chitosan, starch, and a combination thereof.

According to another aspect, provided is a method of synthesizing a polymer in which bovine serum albumin (BSA) is conjugated to a polysaccharide, the method including the steps of: dissolving a mixture of a polysaccharide and BSA, at a concentration of 0.1% (w/v) to 20% (w/v) in distilled water; and inducing hydrothermal synthesis by causing a reaction in a solution in which the polysaccharide and the BSA are dissolved, at 40° C. to 200° ° C. for 6 hours to 72 hours.

The method may further include, after the induction of the hydrothermal synthesis, a step of performing dialysis treatment of the hydrothermally synthesized polymer.

For example, the dialysis treatment step may be performed in distilled water for 24 hours to 72 hours by using a 100-500 molecular weight cut-off (MWCO) dialysis membrane.

Optionally, the method may further include, after the dialysis treatment step, a step of freezing the polymer at a temperature of −20° C. to −70° C., and then freeze-drying the frozen polymer.

In another aspect, provided is an enzyme-stabilizing composition including a polymer in which bovine serum albumin (BSA) is conjugated to a polysaccharide.

The composition may further include a nucleic acid polymerase.

Optionally, the composition may further include at least one selected from a primer, a probe, a deoxyribonucleotide triphosphate (dNTP), and a nucleotide triphosphate (NTP).

If necessary, the composition may further include an additive for a nucleic acid amplification reaction.

In another aspect, provided is a method of amplifying a nucleic acid molecule, the method including the steps of: preparing the above-described enzyme-stabilizing composition; adding a biological sample including the nucleic acid molecule to the enzyme-stabilizing composition; and performing a nucleic acid amplification reaction by using the enzyme-stabilizing composition to which the biological sample has been added.

Before the biological sample is added to the enzyme-stabilizing composition, the enzyme-stabilizing composition may be freeze-dried after being kept frozen at −20° C. to −70° C.

The nucleic acid amplification reaction may be performed using a method selected from the group consisting of polymerase chain reaction (PCR), reverse transcription-polymerase chain reaction (RT-PCR), real-time PCR, reverse transcription, complementary DNA synthesis, loop-mediated isothermal amplification (LAMP), real-time nucleic acid sequence-based amplification (NASBA), strand displacement amplification (SDA), multiple displacement amplification (MDA), rolling circle amplification (RCA), ligase chain reaction (LCR), helicase dependent amplification (HDA), a ramification-extension amplification method (RAM), an in-vitro transcription-based amplification system (TAS), and a combination thereof.

Another aspect provides a method of analyzing the presence of a target nucleic acid molecule in a biological sample, the method comprising the steps of: preparing the above-described enzyme-stabilizing composition; adding a biological sample including the nucleic acid molecule to the enzyme-stabilizing composition; performing a nucleic acid amplification reaction by using the enzyme-stabilizing composition to which the biological sample has been added; and detecting whether the nucleic acid molecule is amplified or not.

### Advantageous Effects of Disclosure

A polymerase chain reaction-stabilizing composition to which a polymer in which bovine serum albumin is conjugated to a polysaccharide, according to the present disclosure, is added stably maintains the activity of a nucleic acid polymerase even when stored at room temperature and high temperature for a long period of time. In particular, unlike a simple mixture of a polysaccharide and bovine serum albumin, the polymer of the present disclosure does not inhibit a reverse transcription (RT) process.

The polymer of the present disclosure and a composition including the same efficiently detect a target nucleic acid molecule associated with the onset of infectious diseases and/or cancer, and thus can be effectively used for the diagnosis of diseases.

### BRIEF DESCRIPTION OF DRAWINGS

FIGS. 1 to 4 are schematic views illustrating a process of synthesizing a polymer in which bovine serum albumin is conjugated to a polysaccharide.

FIG. 5 is a graph showing ¹H-NMR analysis results of a pullulan-bovine serum albumin conjugate synthesized by hydrothermal synthesis, according to an exemplary embodiment of the present disclosure.

FIG. 6 is a graph showing fourier transform infrared spectroscopy (FT-IR) analysis results of a pullulan-bovine serum albumin conjugate synthesized by hydrothermal synthesis, according to an exemplary embodiment of the present disclosure.

FIG. 7 illustrates graphs showing the results of measuring, by using a Zetasizer, the size and the zeta potential of a pullulan-bovine serum albumin conjugate synthesized by hydrothermal synthesis, according to an exemplary embodiment of the present disclosure.

FIG. 8 is a graph showing ¹H-NMR analysis results of a hydroxypropyl cellulose-bovine serum albumin conjugate synthesized by hydrothermal synthesis, according to an exemplary embodiment of the present disclosure.

FIG. 9 is a graph showing FT-IR analysis results of a hydroxypropyl cellulose-bovine serum albumin conjugate synthesized by hydrothermal synthesis, according to an exemplary embodiment of the present disclosure.

FIGS. 10A to 10C are graphs respectively showing the results of measuring the degrees of amplification of target nucleic acid sequences, after biological samples were added to an enzyme-stabilizing composition containing a pullulan-bovine serum albumin conjugate synthesized according to exemplary embodiments of the preset disclosure, and real-time PCR was performed thereon.

FIG. 11 illustrates images showing the results of detecting the presence of a target nucleic acid by using electrophoresis, after a biological sample was added to an enzyme-stabilizing composition containing a pullulan-bovine serum albumin conjugate synthesized according to an exemplary embodiment of the present disclosure, and real-time PCR was performed thereon.

FIG. 12 illustrates images showing the results of detecting the presence of a target nucleic acid by using electrophoresis, after a biological sample was added to an enzyme-stabilizing composition containing a hydroxypropyl cellulose-bovine serum albumin conjugate synthesized according to another exemplary embodiment of the present disclosure, and real-time PCR was performed thereon.

In FIGS. 10A to 10C, 11, and 12, lanes 1 and 2 are the cases of using, after an accelerated aging test, an enzyme-stabilizing composition including a mixture of a polysaccharide and bovine serum albumin, lanes 3 and 4 are the cases of using, after an accelerated aging test, an enzyme-stabilizing composition including the conjugate of the present disclosure, lane 5 is the case of using an enzyme-stabilizing composition kept frozen at −20° C. without the conjugate, and lane 6 is the case of using, after an accelerated aging test, an enzyme-stabilizing composition freeze-dried without the conjugate. In each of lanes 1 to 4 and 6, a nucleic acid amplification reaction was performed after accelerated aging at 40° C. for 14 days.

FIGS. 13 to 18 are graphs showing the results of measuring the degrees of amplification of target nucleic acid sequences, after a biological sample was added to each of an enzyme-stabilizing composition containing a pullulan-bovine serum albumin conjugate and an enzyme-stabilizing composition containing a hydroxypropyl cellulose-bovine serum albumin conjugate, synthesized according to exemplary embodiments of the present disclosure, and an enzyme-stabilizing composition containing Comparative Synthesis Example 1, and real-time PCR was performed thereon.

FIG. 19 illustrates images showing the results of detecting the presence of a target nucleic acid by using electrophoresis, after a biological sample was added to each of an enzyme-stabilizing composition containing a pullulan-bovine serum albumin conjugate and an enzyme-stabilizing composition containing a hydroxypropyl cellulose-bovine serum albumin conjugate, synthesized according to exemplary embodiments of the present disclosure, and an enzyme-stabilizing composition containing Comparative Synthesis Example 1, and real-time PCR was performed thereon.

In FIGS. 13 to 19, lanes 7 and 8 are the cases of using, after an accelerated aging test, an enzyme-stabilizing composition including the conjugate of the present disclosure, lanes 9 and 10 are the cases of using, after an accelerated aging test, an enzyme-stabilizing composition including a conjugate of Comparative Synthesis Example 1, lane 11 is the case of using an enzyme-stabilizing composition kept frozen at −20° C. without the conjugate, and lane 12 is the case of using, after an accelerated aging test, an enzyme-stabilizing composition freeze-dried without the conjugate. In each of lanes 7 to 10 and 12, a nucleic acid amplification reaction was performed after accelerated aging at 40° ° C. for 14 days.

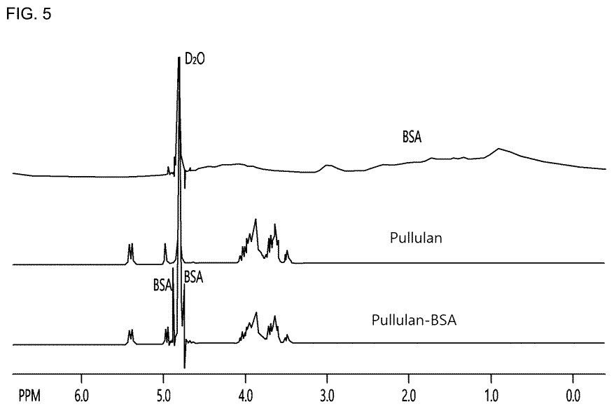

In FIGS. 5 to 19, BSA refers to bovine serum albumin, PUL refers to pullulan, HPC refers to hydroxypropyl cellulose, PUL-BSA refers to a pullulan-bovine serum albumin conjugate, and HPC-BSA refers to a hydroxypropyl cellulose-bovine serum albumin conjugate.

### MODE OF DISCLOSURE

Hereinafter, the present disclosure will be described with reference to the accompanying drawings as necessary.

### Definition of Terms

The term “amino acid” as used herein is used in the broadest sense and is intended to include naturally-occurring L-amino acids or residues thereof. Amino acids include not only D-amino acids but also chemically-modified amino acids, for example, amino acid analogues, naturally-occurring amino acids that are not typically incorporated to proteins, for example, norleucine, and chemically-synthesized compounds having properties known in the art. For example, phenylalanine or proline analogues or mimics that allow conformational restriction of the same peptide compound as natural Phe or Pro are included within the definition of amino acids. Such analogues and mimics are referred to herein as “functional equivalents” of amino acids.

For example, synthetic peptides synthesized by standard solid-phase synthetic techniques are not limited to amino acids encoded by a gene, thus allowing a wider variety of substitutions for the given amino acid. Amino acids not encoded by the genetic code are referred to herein as “amino acid analogues.”

For example, amino acid analogues include: 2-amino adipic acid (Aad) for Glu and Asp; 2-aminopimelic acid (Apm) for Glu and Asp; 2-aminobutyric acid (Abu) for Met, Leu, and other aliphatic amino acids; 2-aminoheptanoic acid (Ahe) for Met, Leu, and other aliphatic amino acids; 2-aminoisobutyric acid (Aib) for Gly; cyclohexylalanine (Cha) for Val, Leu, and Ile; homoarginine (Har) for Arg and Lys; 2,3-diaminopropionic acid (Dap) for Lys, Arg, and His; N-ethylglycine (EtGly) for Gly, Pro, and Ala; N-ethylglycine (EtGly) for Gly, Pro, and Ala; N-ethylasparagine (EtAsn) for Asn and Gln; hydroxyllysine (Hyl) for Lys; allohydroxyllysine (AHyl) for Lys; 3-(and 4-)hydoxyproline (3Hyp, 4Hyp) for Pro, Ser, and Thr; allo-isoleucine (Alle) for Ile, Leu, and Val; 4-amidinophenylalanine for Arg; N-methylglycine (MeGly, sarcosine) for Gly, Pro, and Ala; methylisoleucine (Melle) for Ile; norvaline (Nva) for Met and other aliphatic amino acids; norleucine (Nle) for Met and other aliphatic amino acids; ornithine (Om) for Lys, Arg, and His; citrulline (Cit) and methionine sulfoxide (MSO) for Thr, Asn, and Gln; and N methylphenylalanine (MePhe), trimethylphenylalanine, halo-(F-, Cl-, Br-, or I-)phenylalanine, or trifluorylphenylalanine, for Phe.

The term “peptide” as used herein includes all proteins, protein fragment and peptides isolated from naturally occurring ones or synthesized by recombinant techniques or chemically. For example, the peptide of the present disclosure may consist of at least 5, for example, at least 10 amino acids.

In specific embodiments, compound variants, such as peptide variants having one or more amino acid substitutions, is provided. The term “peptide variants” as used herein refers to peptides of which the amino acid sequence has one or more amino acid substitutions, deletions, additions and/or insertions, and ones that exhibit almost the same biological function as peptide consisting of original amino acids. A peptide variant should have at least 70%, preferably at least 90%, and more preferably at least 95% identity with an original peptide. Such substituents may include amino acid substituents known as “conservative.” Variants may also include nonconservative changes. In exemplary embodiments, the sequence of a variant polypeptide differs from the original sequence by substitution, deletion, addition, or insertion of five or fewer amino acids. Variants may also be altered by deletion or addition of amino acids that have minimal effect on the immunogenicity, secondary structure and hydropathic nature of a peptide.

Conservative substitution means that there is no significant change in properties such as the secondary structure and hydropathic nature of a polypeptide even when one amino acid is substituted with another amino acid. Amino acid mutations may be obtained on the basis of the relative similarity of amino acid side chain substituents, such as similarity in polarity, charge, solubility, hydrophobicity, hydrophilicity, and/or amphipathic nature.

For example, amino acids may be classified according to common side chain properties as follows: i) hydrophobic (norleucine, methionine, alanine, valine, leucine, and isoleucine); ii) neutral hydrophilic (cysteine, serine, threonine, asparagine, and glutamine); iii) acidic (aspartic acid and glutamic acid); iv) basic (histidine, lysine, and arginine); v) residues that affect chain orientation (glycine and proline); and vi) aromatic (tryptophan, tyrosine, and phenylalanine). Conservative substitutions will involve exchanging a member of one of these respective classes for another member of the same class.

Through analysis of the sizes, shapes and types of amino acid side chain substituents, it can be seen that arginine, lysine, and histidine are all positively charged residues, alanine, glycine, and serine have similar sizes, and phenylalanine, tryptophan, and tyrosine have similar shapes. Thus, based on these considerations, arginine, lysine, and histidine may be biologically functional equivalents, alanine, glycine, and serine may be biologically functional equivalents, and phenylalanine, tryptophan, and tyrosine may be biologically functional equivalents.

In introducing mutations, the hydropathic index of amino acids can be considered. Each amino acid is assigned a hydrophobicity index according to the hydrophobicity and charge thereof: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

The hydrophobic amino acid index is very important in imparting the interactive biological function of proteins. It is known that substitution with amino acids having a similar hydrophobic index can retain similar biological activities. When a mutation is introduced with reference to the hydrophobic index, the substitution is made between amino acids showing a hydrophobic index difference preferably within ±2, more preferably within ±1, and even more preferably within ±0.5.

Meanwhile, it is also well known that the substitution between amino acids with similar hydrophilicity values leads to proteins with equivalent biological activity. The following hydrophilicity value is assigned to each amino acid residue: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); lysine (−1.8); isoleucine (−1.8); tyrosine (−2.3); phenylalanine (−2.5); and tryptophan (−3.4). When a mutation is introduced with reference to the hydrophilicity value, the substitution is made between amino acids showing a hydrophilicity value difference preferably within ±2, more preferably within ±1, and even more preferably within ±0.5.

Amino acid substitution in proteins that do not totally alter the activity of a molecule is known in the art (H. Neurath, R. L. Hill, The Proteins, Academic Press, New York, 1979). The substitution occurs the most commonly between amino acid residues, e.g., Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

In general, the peptides (including fusion proteins) and polynucleotides referred to herein are isolated. An isolated peptide or polynucleotide is one that has been removed from its original environment. For example, a protein that exists in its natural state is isolated by removing all or part of the substances present together in that state. Such a polypeptide should have a purity of at least 90%, preferably at least 95%, and more preferably at least 99%. Polynucleotides are isolated by cloning in a vector.

Peptides may be isolated by recombinant means or by preparation through chemical synthesis. Recombinant peptides encoded by the nucleotide sequences mentioned herein, for example, peptides consisting of the amino acid sequence(s) of SEQ ID NO: 1 and/or SEQ ID NO: 2, can be easily prepared using a known method regardless of whether any of many known expression vectors is used. The recombinant peptide may be expressed in a suitable host cell transformed with an expression vector including a DNA sequence encoding a recombinant protein. Suitable host cells include prokaryotes, yeast, and eukaryotes. For example, it is preferable to use a cell line derived from a eukaryote such as yeast, an insect cell, or a mammalian cell line (such as Cos or CHO), as the host cell.

To purify the recombinant protein, first, a supernatant obtained from an aqueous host/vector system and including a recombinant protein secreted into a culture medium is concentrated using a commercially available filter. In the next step, the obtained concentrate is purified using a suitable purification matrix such as an affinity matrix or an ion exchange resin. Finally, one or more steps of reverse phase HPLC may be performed to obtain a pure recombinant protein.

The peptides or proteins described herein may be secreted into and recovered from the periplasm of host cells. Peptide and/or protein recovery typically involves disrupting a microorganism, generally by means such as osmotic shock, sonication, or lysis. Once cells are disrupted, cell debris or whole cells may be removed by centrifugation or filtration. Peptides and/or proteins may be further purified, for example, by affinity resin chromatography.

Alternatively, peptides and/or proteins may be transported into culture media and isolated therefrom. Cells may be removed from the culture, and peptides and/or proteins produced by filtering and concentrating the culture supernatant may be further purified. The expressed polypeptides may be further isolated and identified using commonly known methods, such as: fractional distillation on immunoaffinity or ion-exchange columns; ethanol precipitation; reverse phase HPLC; chromatography on silica or cation exchange resins such as DEAE; chromatofocusing; SDS-PAGE; ammonium sulfate precipitation; gel filtration using, for example, Sephadex G-75; ligand affinity using hydrophobic affinity resins and suitable antigens immobilized on a matrix; and Western blot assays.

The term “polynucleotide” or “nucleic acid” as used herein is interchangeably used, refers to polymers of nucleotides of any length, and comprehensively includes DNA (e.g., cDNA) and RNA molecules. The term “nucleotides,” which are the structural unit of nucleic acid molecules, may refer to deoxyribonucleotides, ribonucleotides, modified nucleotides or bases, and/or their analogues, or any substrate that can be incorporated into a polymer by DNA or RNA polymerase, or by a synthetic reaction. Polynucleotides may include modified nucleotides, and sugar- or base-modified analogues, such as methylated nucleotides and analogues thereof.

A mutation in a nucleotide may not result in a mutation in a protein. Such a nucleic acid may include a nucleic acid molecule including functionally equivalent codons or codons encoding the same amino acid (for example, due to degeneracy of codons, there are six codons for arginine or serine), or codons encoding a biologically equivalent amino acid. Also, a mutation in a nucleotide may result in a change in a protein itself. Even in the case of a mutation that causes a change in the amino acid of a protein, one exhibiting almost the same activity as the protein of the present disclosure may be obtained.

Within the range of the properties of the nucleic acid molecule or polynucleotide of the present disclosure, for example, the effect thereof as a vaccine and/or an adjuvant, it is obvious to those of ordinary skill in the art that the peptide and nucleic acid molecule of the present disclosure is not limited to the amino acid sequences or nucleotide sequences listed in the sequence list. For example, a coding region operably linked to an expression regulation sequence and/or a biologically functional equivalent that may be included in a recombinant protein/peptide expressed therefrom may be a polynucleotide having a mutation in the base sequence and/or a protein/peptide having a mutation in the amino acid sequence, which exhibit(s) biological activity equivalent to that of the above-described coding region and/or recombinant protein.

In consideration of the above-described mutation having biological equivalent activity, it is construed that a nucleic acid molecule encoding the peptide and/or protein also includes a sequence exhibiting substantial identity to the sequence described in the sequence list. The substantial identity refers to a sequence having at least 61% homology, more preferably at least 70% homology, even more preferably at least 80% homology, and most preferably at least 90% homology, when the sequence of the present disclosure and any other sequences are aligned to correspond to each other as much as possible, and the aligned sequence is analyzed using an algorithm commonly used in the art to which the present disclosure pertains. Alignment methods for sequence comparison are known in the art to which the present disclosure pertains.

The term “primers” as used herein refers to single-stranded oligonucleotides which are respectively complementary to the 5′-terminal sequence and/or the 3′-terminal sequence on a target nucleic acid molecule to be amplified in a nucleic acid amplification reaction, and are capable of acting as the points of initiation for a polymerase chain reaction between template-directed nucleic acids under suitable conditions (e.g., four different (di)nucleoside triphosphates and a polymerase) in a suitable buffer at a suitable temperature. The appropriate length of the primer may vary depending on various factors, for example, temperature and the intended use, but the primer may generally consist of 15 to 50 nucleotides.

The term “probe” as used herein refers to a single-stranded nucleic acid molecule, and an oligonucleotide complementary to a target nucleic acid sequence under stringent conditions. For example, the probe may consist of 10 to 20 nucleotides.

The term “hybridization under stringent conditions” as used herein means that two single-stranded nucleic acid molecules consist of at least 70%, for example, at least 80% or at least 90% of complementary nucleotides.

The inventors of the present disclosure researched a method in which enzyme activity can be stably maintained even when stored for a long period of time at room temperature and high temperature in relation to nucleic acid amplification. Accordingly, it was discovered that, when a polysaccharide-bovine serum albumin (BSA) conjugate was added to a nucleic acid amplification reaction-stabilizing composition, the stability of a nucleic acid amplification enzyme could be maintained.

In one aspect, the present disclosure provides a polymer in which bovine serum albumin (BSA) is conjugated to a polysaccharide. According to the present disclosure, even when an enzyme-stabilizing composition containing the polymer in which bovine serum albumin is conjugated to a polysaccharide is stored for a long period of time, the activity of a nucleic acid polymerase is stably maintained.

The bovine serum albumin-conjugated polysaccharide is not particularly limited as long as the polysaccharide can have any glycosidic bond between monosaccharides as unit components. For example, the polysaccharide may be selected from, but is not limited to, the group of consisting of cellulose, hydroxypropyl cellulose, hydroxypropyl methylcellulose, glycosaminoglycan, pullulan, alginic acid, carrageenan, aribinogalactan, hemicellulose, dextran, chitosan, glycol chitosan, starch, and a combination thereof. In an exemplary aspect, the polysaccharide may be selected from the group consisting of cellulose, hydroxypropyl cellulose, hydroxypropyl methylcellulose, pullulan, and a combination thereof.

The polymer in which bovine serum albumin is conjugated to a polysaccharide may be synthesized through a hydrothermal reaction between the polysaccharide and the bovine serum albumin. More specifically, the synthesis method includes the steps of: dissolving a mixture of a polysaccharide and bovine serum albumin (BSA), at a concentration of 0.1% (w/v) to 20% (w/v) in distilled water; and inducing hydrothermal synthesis by causing a reaction in a solution in which the polysaccharide and the bovine serum albumin are dissolved, at 40° C. to 200° ° C. for 6 hours to 72 hours, and optionally, may further include the steps of: after the hydrothermal synthesis, performing dialysis treatment of the solution in which the polymer is produced; and freezing the synthesized polymer at a temperature of −20° C. to −70° C., followed by freeze-drying.

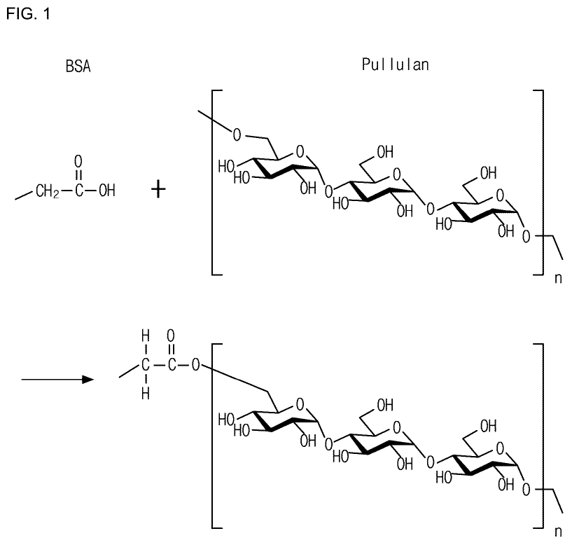

FIGS. 1 to 4 are schematic views illustrating a process of synthesizing, by hydrothermal synthesis, a polymer in which bovine serum albumin is conjugated to pullulan as an example of a polysaccharide, according to exemplary embodiments of the present disclosure. Although FIGS. 1 to 4 illustrate pullulan as a polysaccharide, bovine serum albumin may be conjugated to any polysaccharide having a glycosidic bond. In addition, although FIGS. 1 to 4 illustrate the case in which bovine serum albumin is conjugated to the glycoside-binding site of a polysaccharide, bovine serum albumin is not necessarily conjugated to the glycoside-binding site of a polysaccharide.

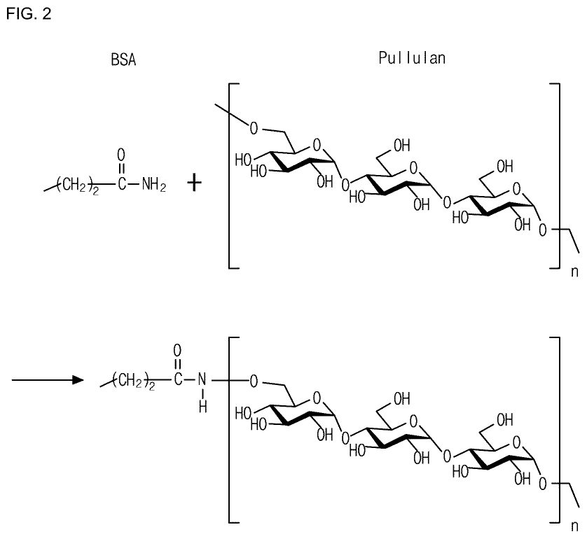

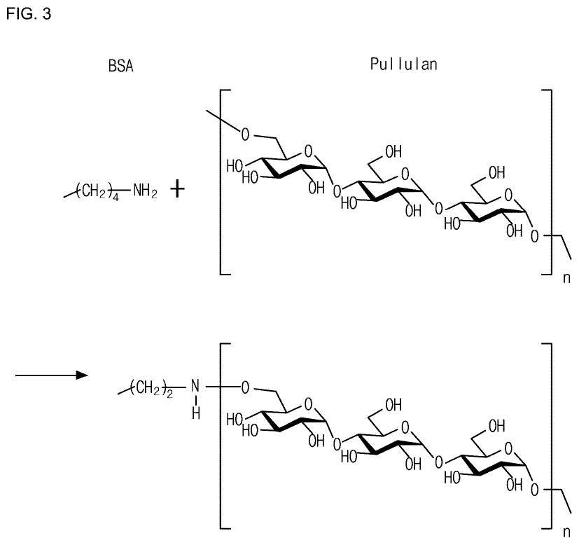

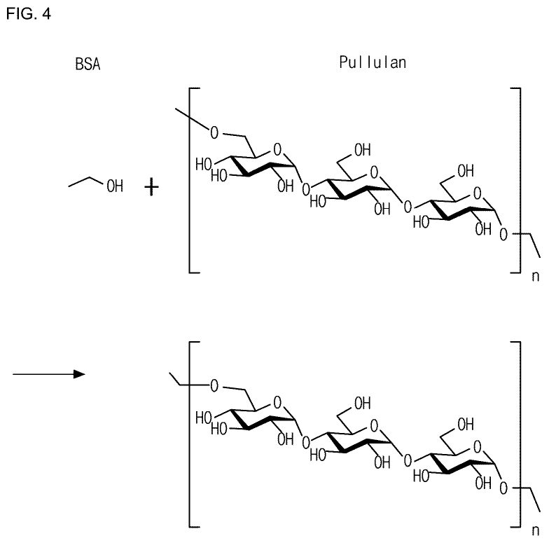

In FIG. 1, a carboxylic acid group, which is part of a terminal and/or a specific amino acid side chain of bovine serum albumin, is conjugated to the glycoside-binding site of pullulan. In this case, bovine serum albumin and pullulan may be linked through an ester bond. In FIG. 2, an amide group, which is part of a terminal and/or an amino acid side chain of bovine serum albumin, is conjugated to the glycoside-binding site of pullulan. In this case, bovine serum albumin and pullulan may be linked via an N-acetylglucozamine form, i.e., an N-linked glycosidic bond. Optionally, in FIG. 3, an amino group, which is part of an amino acid side chain constituting bovine serum albumin, is conjugated to the glycoside-binding site of pullulan. In addition, in FIG. 4, a hydroxyl group, which is part of an amino acid side chain constituting a bovine serum amino acid, is conjugated to the glycoside-binding site of pullulan.

For example, the content of bovine serum albumin in the polymer in which bovine serum albumin is conjugated to a polysaccharide may be 0.01 wt % to 70 wt %, for example, 10 wt % to 70 wt % or 40 wt % to 60 wt %. When the content of bovine serum albumin in the polymer is less than 0.01 wt %, the content of bovine serum albumin conjugated to a polysaccharide is too small to expect an enzyme-stabilizing effect by conjugation of bovine serum albumin. On the other hand, when the content of bovine serum albumin in the polymer exceeds 70 wt %, bovine serum albumin affects the reverse transcription of a polymerase, and thus, the activity of the polymerase may be reduced.

After the hydrothermal synthesis is completed, the hydrothermally synthesized polymer may be subjected to dialysis treatment. For example, the dialysis treatment step may be performed in distilled water by using a 100-500 molecular weight cut-off (MWCO) dialysis membrane for 24 hours to 72 hours. Optionally, the pH of the solution in the dialysis treatment step may be 5 to 9.

In another alternative aspect, after the dialysis treatment, the polymer in which bovine serum albumin is conjugated to a polysaccharide may be frozen at a temperature of −20° C. to −70° C., and then freeze-dried.

As described above, the polymer in which bovine serum albumin is conjugated to a polysaccharide stabilizes an enzyme, such as a nucleic acid polymerase. Accordingly, in another aspect, the present disclosure relates to an enzyme-stabilizing composition including a polymer in which bovine serum albumin is conjugated to a polysaccharide.

In an exemplary aspect, in the enzyme-stabilizing composition, the polymer in which bovine serum albumin is conjugated to a polysaccharide may be included at a concentration of 0.01% (w/v) to 10% (w/v), for example, 0.5% (w/v) to 5% (w/v) or 0.5% (w/v) to 3% (w/v). When the content of the polymer in which bovine serum albumin is conjugated to a polysaccharide in the enzyme-stabilizing composition is less than 0.01% (w/v), it is difficult to realize the effect of stabilizing enzymatic activity. When the content of the polymer in which bovine serum albumin is conjugated to a polysaccharide in the enzyme-stabilizing composition exceeds 10% (w/v), the intended reaction such as a nucleic acid amplification reaction may not be efficiently performed while the contents of other components are reduced.

DNA and/or RNA polymerase that may be added in the enzyme-stabilizing composition is not particularly limited as long as the amplification reaction of a target nucleic acid molecule can be performed using the corresponding polymerase. The DNA polymerase may include a Klenow fragment of E. coli DNA polymerase I, thermostable DNA polymerase, and bacteriophage T7 DNA polymerase. In an exemplary aspect, the polymerase, which is a thermostable DNA polymerase obtainable from a variety of bacterial species, includes, but is not limited to, Thermus aquaticus(Taq), Thermus thermophilus(Tth), Thermus filiformis, Thermis flavus, Thermococcus literalis, and/or Pyrococcus furiosus(Pfu).

For example, a commercially available premix form of a polymerase and dNTP may be used in the enzyme-stabilizing composition. In this case, the mixture of a polymerase and dNTP may be included in the enzyme-stabilizing composition at a concentration of 5% (w/v) to 40% (w/v), for example, 10% (w/v) to 30% (w/v) or 15% (w/v) to 25% (w/V).

As an example of the enzyme-stabilizing composition, a nucleic acid amplification reaction solution may include a dNTP mixture (dATP, dCTP, dGTP, dTTP), a nucleic acid amplification reaction buffer, other nucleic acid amplification reaction additives, and a nucleic acid polymerase cofactor. When a nucleic acid amplification reaction is performed, excess amounts of components needed for the reaction may be supplied to a reaction vessel. The excess amounts of components needed for the nucleic acid amplification reaction refer to amounts such that the amplification reaction is not substantially limited to the concentrations of the components. A cofactor, such as Mg²⁺, and dNTP may be supplied to a reaction solution to an extent to which a desired amplification reaction can be achieved.

For example, in the nucleic acid amplification reaction, annealing is performed under stringent conditions that enhance specific binding between the nucleotide sequence of a target nucleic acid molecule and a primer sequence. The term “annealing” or “priming” refers to the apposition of an oligonucleotide or a nucleic acid to a template nucleic acid molecule, and accordingly, a polymerase polymerizes nucleotides to form a nucleic acid molecule complementary to a template nucleic acid molecule or a fragment thereof. Stringent conditions for annealing are sequence-dependent and vary according to environmental variables. In an exemplary aspect, the primer may be included in the enzyme-stabilizing composition at a concentration of 5% (w/v) to 40% (w/v), for example, 5% (w/v) to 30% (w/v) or 5% (w/v) to 20% (w/v).

If necessary, the enzyme-stabilizing composition may further include an additive for a nucleic acid amplification reaction. For example, the additive for a nucleic acid amplification reaction may be selected from the group consisting of mannitol, polyethylene glycol (e.g., PEG 10,000), trehalose, betaine, and a combination thereof. In this case, the additive for a nucleic acid amplification reaction may be added to the enzyme-stabilizing composition at a concentration of 0.5% (w/v) to 30% (w/v), for example, 0.5% (w/v) to 20% (w/v) or 1% (w/v) to 15% (w/V).

The enzyme-stabilizing composition may include a suitable buffer for the nucleic acid amplification reaction. The type of the buffer is not particularly limited, but may be selected from the group consisting of organic acids, glycine, histidine, glutamate, succinate, phosphate, acetate, citrate, tris (e.g., tris-EDTA), hydroxyethyl piperazine ethane sulfonic acid (HEPES), amino acids, and a combination thereof.

In the method of amplifying a nucleic acid and the method of detecting a target nucleic acid molecule, according to the present disclosure, the amplified target sequence may be labeled with a detectable marker. In one exemplary aspect, the marker may be, but is not limited to, a substance that emits fluorescence, phosphorescence, chemiluminescence, or radioactivity. For example, the marker may be fluorescein, phycoerythrin, rhodamine, lissamine. Cy-5, or Cy-3.

In the case in which a target nucleic acid is amplified, when the 5′-terminal and/or the 3′-terminal of primer(s) is(are) labeled with Cy-5 or Cy-3, and a nucleic acid amplification reaction (e.g., real-time polymerase chain reaction) is performed, the target nucleic acid may be labeled with a detectable fluorescent marker. In addition, for labeling using a radioactive material, when a nucleic acid amplification reaction (e.g., real-time polymerase chain reaction) is performed, by adding a radioactive isotope such as P³² and/or S³⁵ to the enzyme-stabilizing composition according to the present disclosure, the radioactive material is incorporated into the amplified product while the amplified product is synthesized, so that the amplified product can be radioactively labeled. One or more oligonucleotide primer sets may be used to amplify a target nucleic acid.

For labeling, various methods commonly performed in the art to which the present disclosure pertains may be used. For example, labeling may be performed through a nick translation method, a random priming method (Multiprime DNA labelling systems booklet, “Amersham” (1989)), and a kynation method (Maxam & Gilbert, Methods in Enzymology, 65:499(1986)). The marker provides a signal which can be detected by fluorescence, radioactivity, phosphorescence, colorimetry, gravimetry, X-ray diffraction or absorption, magnetism, enzymatic activity, mass analysis, binding affinity, hybridization radiofrequency, or nanocrystals.

A primer and/or a probe may specifically bind to a target nucleic acid molecule present in a biological sample. For example, the target nucleic acid molecule may be derived from cancer, a tumor, or a pathogen, and may be in the form of DNA or RNA. For example, cancers or tumors include, but are not limited to, stomach cancer, lung cancer, liver cancer, colorectal cancer, small intestine cancer, skin cancer, pancreatic cancer, and prostate cancer.

Optionally, pathogens may include, but are not limited to, pathogenic viruses (e.g., human papillomavirus (HPV), influenzavirus, coronavirus, and the like) and pathogenic bacteria (pneumococcus and the like). The target nucleic acid molecule may be a nucleic acid molecule specific for cancer, a tumor or a pathogen, or a nucleic acid molecule as a marker indicating whether cancer or a tumor is developed or whether to be infected with a pathogen.

If necessary, before a biological sample including the target nucleic acid molecule is added to the enzyme-stabilizing composition, the enzyme-stabilizing composition may be kept frozen at −20° ° C. to −70° C., and then freeze-dried. In this case, the polymer in which bovine serum albumin is conjugated to a polysaccharide may be rehydrated after freeze-drying, and then added to the enzyme-stabilizing composition along with an additive for a nucleic acid amplification reaction.

The nucleic acid amplification reaction is not particularly limited as long as the reaction can amplify a target nucleic acid molecule that may be present in a biological sample. For example, the nucleic acid amplification reaction may be performed using a method selected from the group consisting of polymerase chain reaction (PCR), reverse transcription-polymerase chain reaction (RT-PCR), real-time PCR, reverse transcription, complementary DNA synthesis, loop-mediated isothermal amplification (LAMP), real-time nucleic acid sequence-based amplification (NASBA), strand displacement amplification (SDA), multiple displacement amplification (MDA), rolling circle amplification (RCA), ligase chain reaction (LCR), helicase-dependent amplification (HDA), a ramification-extension amplification method (RAM), an in-vitro transcription-based amplification system (TAS), and a combination thereof.

Hereinafter, the present disclosure will be described with reference to exemplary examples, but is not limited to the technical ideas described in the following examples.

### Synthesis Example 1: Synthesis of Conjugate of Pullulan and Bovine Serum Albumin

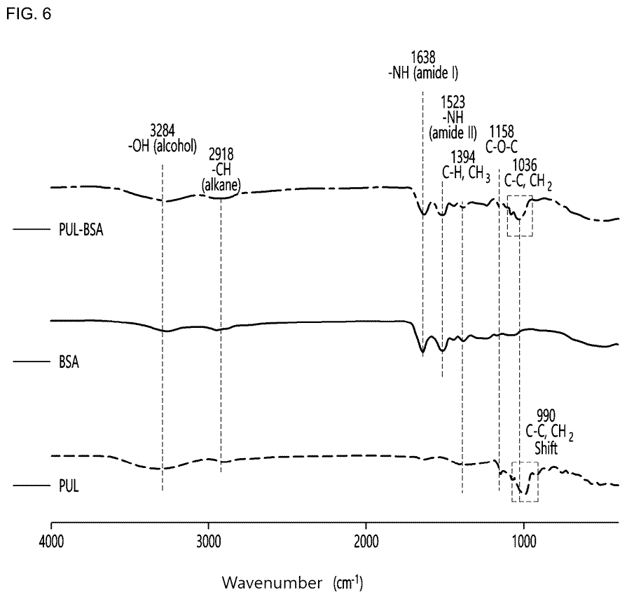

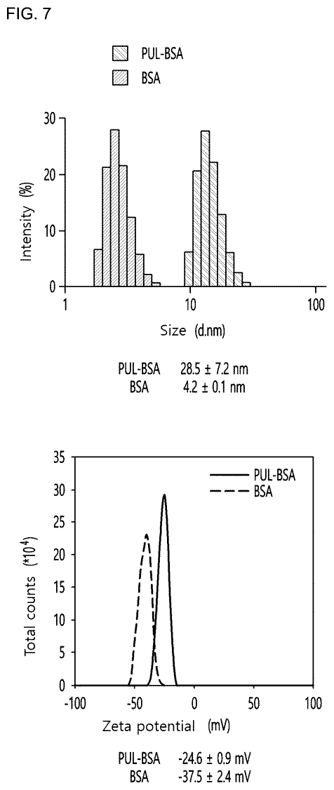

100 mg of pullulan and 1-200 mg of bovine serum albumin (BSA) were dissolved in 100 ml of distilled water, and then the solution was put in a mold coated with 50 ml of Teflon to cause a reaction at 40° C. to 200° ° C. for 6 hours to 72 hours, thereby obtaining a primary synthesized product. The obtained primary synthesized product was dialyzed for 3 days in distilled water at a pH of 5.0 to 9.0 using a 100-500 molecular weight cut-off (MWCO) dialysis membrane. The dialyzed synthesized product was freeze-dried to recover a polymer for enzyme stabilization. The 1H NMR analysis results of a polymer in which BSA is conjugated to pullulan are illustrated in FIG. 5, the FT-IR analysis results thereof are illustrated in FIG. 6, and the results of analyzing the size and the zeta potential of the polymer by using a Zetasizer are illustrated in FIG. 7.

### Synthesis Example 2: Synthesis of Conjugate of Hydroxypropyl Cellulose and Bovine Serum Albumin

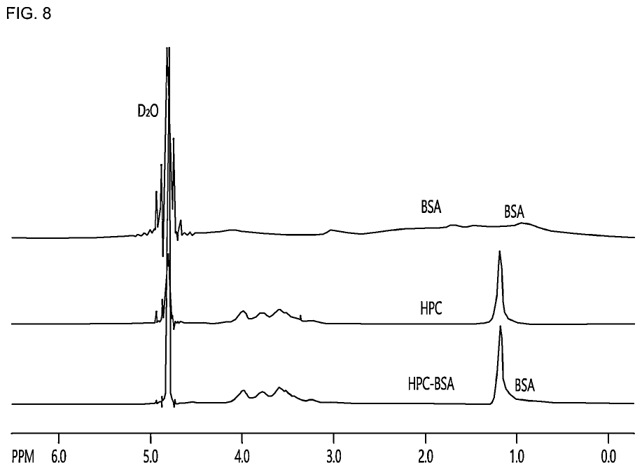

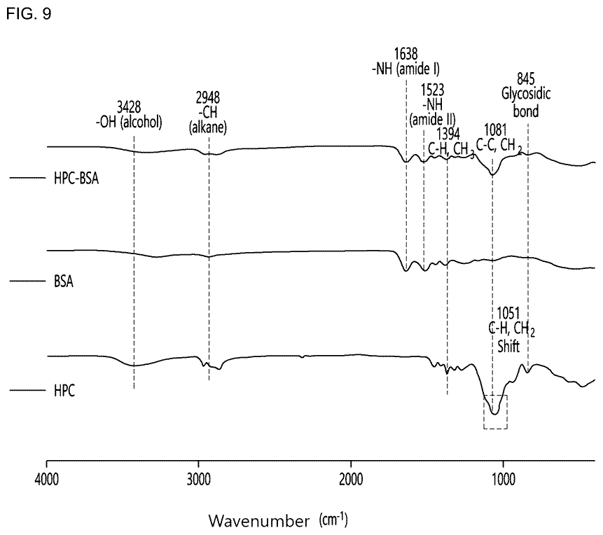

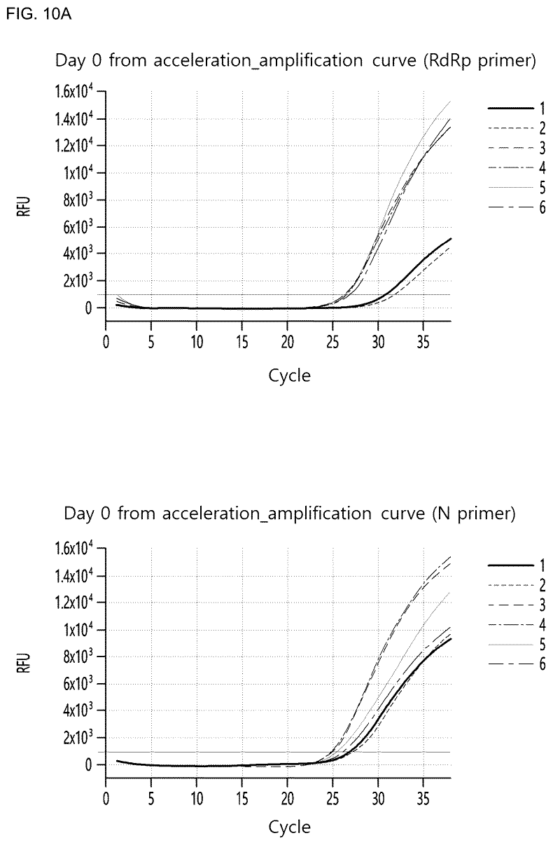

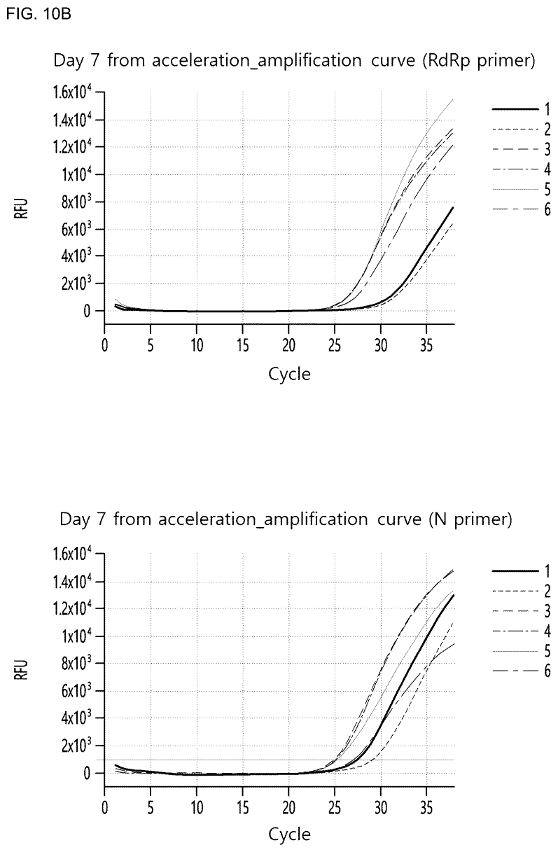

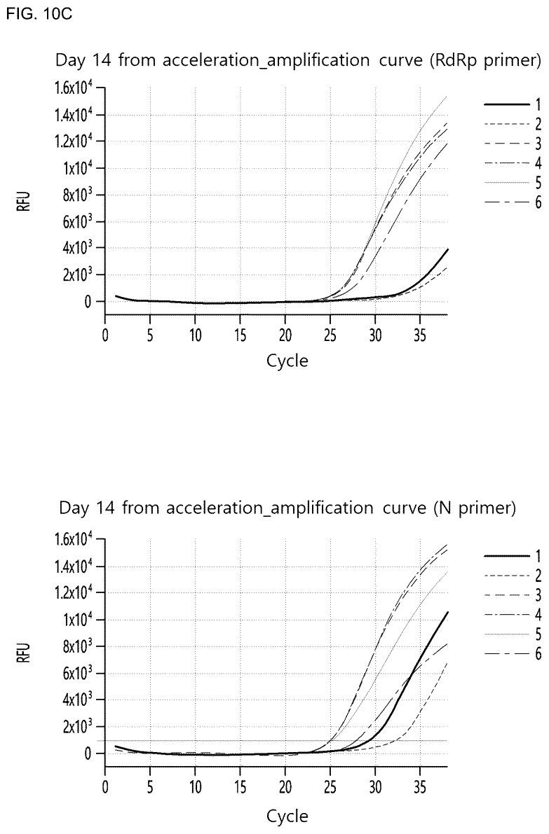

A polymer in which BSA is conjugated to hydroxypropyl cellulose (HPC) was synthesized in the same manner as in Synthesis Example 1, except that 100 mg of HPC was used as a starting material instead of pullulan. The 1H NMR analysis results of the polymer in which BSA is conjugated to HPC are illustrated in FIG. 8, and the FT-IR analysis results thereof are illustrated in FIG. 9.

### Comparative Synthesis Example 1: Synthesis of Conjugate of Dextran and Bovine Serum Albumin

As disclosed in the literature [Sonali S. Rohiwal et al., ‘Self-assembly of bovine serum albumin (BSA)-dextran bio-nanoconjugate: structural, antioxidant and in vitro wound healing studies’, RSC Adv., 2021, 11, 4308-4317 (Jan. 21, 2021)], a conjugate of dextran and bovine serum albumin was synthesized by the Maillard reaction between dextran and BSA for 7 days in accordance with the above literature.

### Example 1: Preparation of Enzyme-Stabilizing Composition

A nucleic acid amplification reaction-stabilizing composition was prepared as an example of an enzyme-stabilizing composition including the polymer of Synthesis Example 1 in which BSA is conjugated to pullulan. Components added to the nucleic acid amplification reaction-stabilizing composition and the contents thereof are shown in Table 1 below. Specifically, 10 μl of the nucleic acid amplification reaction-stabilizing composition was dispensed into 0.2 ml of a PCR tube or a 96-well plate, frozen at −20° C. for 24 hours, and then freeze-dried for 24 hours to 72 hours to be crystallized. The crystallized nucleic acid amplification reaction-stabilizing composition was subjected to an accelerated aging test at 40° C. for two weeks.

[symbol] Announcement of Establishment of Medical Device Stability Test Standards [symbol] was referred to for the accelerated aging test, and the accelerated aging factor (AAF) was measured according to the following expression.

$$AAF = {Q}_{{10}^{[ ( TAA - TRT ) / 10 ]}}$$

wherein TAA is an accelerated aging temperature (° C.), TRT is a product storage temperature (° C.), and accelerated aging time (AAT) is set (RT)/AAF

After exposure at a temperature of 40° C. for 0, 7 and 14 days, experiments were carried out. When converted on the basis of −20° C., 7 days and 14 days mean that stability can be maintained for 448 days and 896 days, respectively.

Meanwhile, to establish a control, pullulan and BSA were simply mixed to prepare a nucleic acid amplification reaction-stabilizing composition, and the accelerated aging test was carried out according to the same process. In addition, other controls were classified as a group in which liquid Taq polymerase was stored at −20° C., and a group in which a buffer is added, followed by freeze-drying and storage at 40° C. for two weeks, to perform the accelerated aging test.

TABLE 1
Components of Nucleic Acid Amplification Reaction-Stabilizing Composition

| | Group | | | | | |
|---|---|---|---|---|---|---|
| | Pullulan + BSA | | Conjugated Polymer | | Frozen | Freeze-drying |
| | (40° C.) | | (40° C.) | | (−20° C.) | (40° C.) |
| Classification | 1 | 2 | 3 | 4 | 5 | 6 |
| Polymer (5%) | 4 μl | 6 μl | 4 μl | 6 μl | — | — |
| Premix* | 4 μl | 4 μl | 4 μl | 4 μl | 4 μl | 4 μl |
| Additive* | 5 μl | 5 μl | 5 μl | 5 μl | — | — |
| TE buffer | 7 μl | 5 μl | 7 μl | 5 μl | — | 16 μl |
| Total volume | 20 μl | 20 μl | 20 μl | 20 μl | 4 μl | 20 μl |

Premix: Mix Taq polymerase (5 unit/μl) and 2.5 mM dNTP
Additives: Mannitol, PEG 10000, Trehalose, and Betaine (5 wt % to 40 wt %)

### Experimental Example 1: Evaluation of Nucleic Acid Amplification Performance

To evaluate the polymerase stabilization performance of the nucleic acid amplification reaction-stabilizing composition prepared according to Example 1, a sample in which a positive sample of a corona-19 virus (COVID-19) patient was diluted to 1/100, and forward and reverse primers specific to an RNA dependent RNA polymerase (RdRP) region and a nucleocapsid (N) region in the open reading frame (OFR) of corona-19 virus were added to the nucleic acid amplification reaction-stabilizing composition stored after the accelerated aging test, and then a nucleic acid amplification reaction was performed. 10 μl of positive sample RNA, 4 μl of the primer specific to an RdRP region, 4 μl of the primer specific to an N region, and 0.1 μl of an internal control were added. For the nucleic acid amplification reaction, 1 cycle was performed at 50° C. for 5 minutes and at 94° C. for 20 seconds, followed by 38 cycles at 90° C. for 1 second and at 64° C. for 10 seconds.

The results of measuring the degree of amplification in the nucleic acid amplification reaction according to the accelerated aging time are illustrated in FIGS. 10A to 10C. The samples of lanes 3 and 4 to which the polymer according to the present disclosure in which BSA is conjugated to pullulan was added had little difference in the activity of a polymerase, compared with lane 5 as a control in the frozen state. Meanwhile, it was confirmed that the samples of lanes 1 and 2 to which a simple mixture of pullulan and BSA was added affected the reverse transcription of a polymerase, and thus, the activity of the polymerase was remarkably reduced.

### Experimental Example 2: Evaluation of Nucleic Acid Amplification Performance by Using Electrophoresis

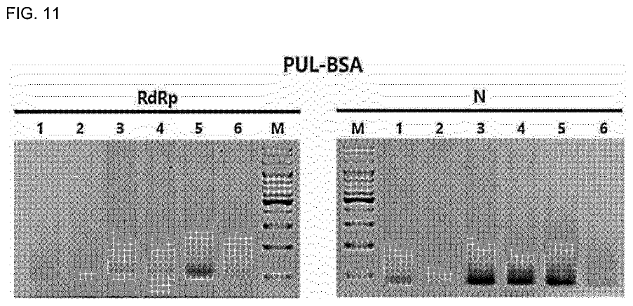

After a target nucleic acid molecule in each sample was amplified using the nucleic acid amplification reaction in Experimental Example 1, electrophoresis (100 V, 20 minutes) was performed on an agarose gel. The electrophoretic analysis results are illustrated in FIG. 11. As in Experimental Example 1, the polymer according to the present disclosure in which BSA is conjugated to pullulan remarkably improved the stability of a polymerase, whereas the activity of the polymerase was greatly reduced in the sample in which pullulan and BSA were simply mixed.

### Experimental Example 3: Evaluation of Nucleic Acid Amplification Performance by Using Electrophoresis

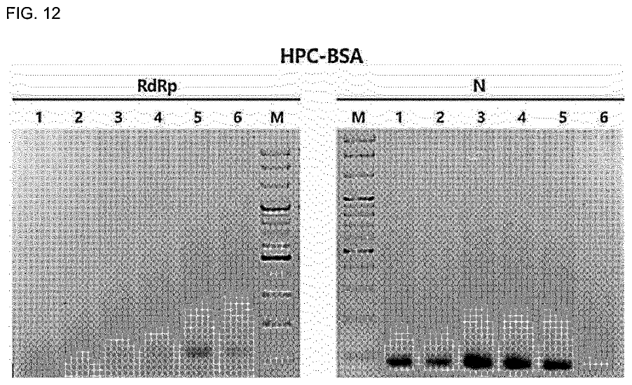

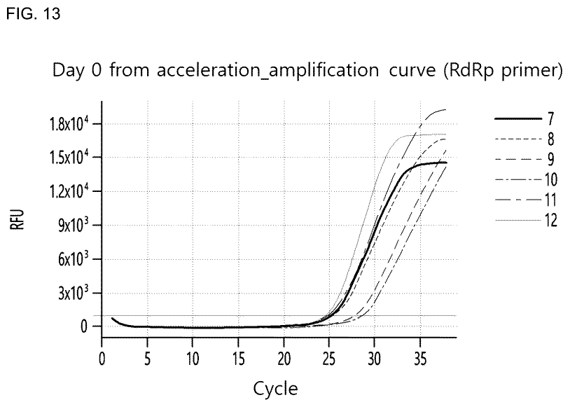

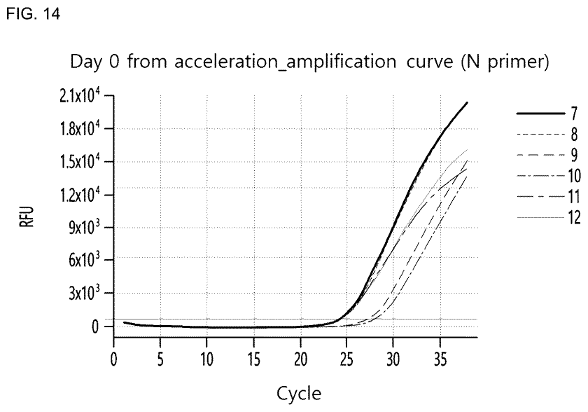

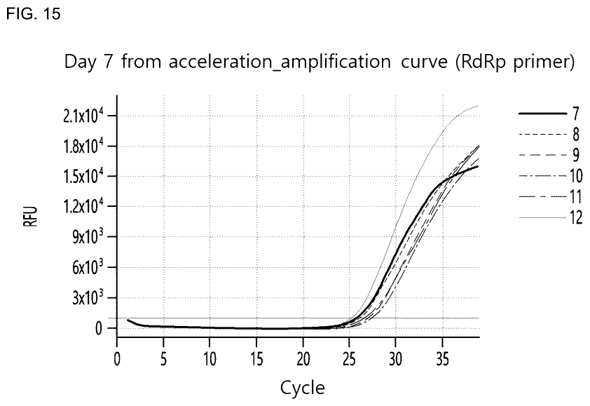

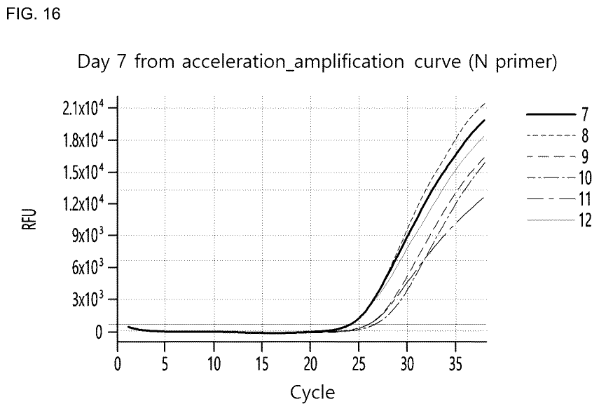

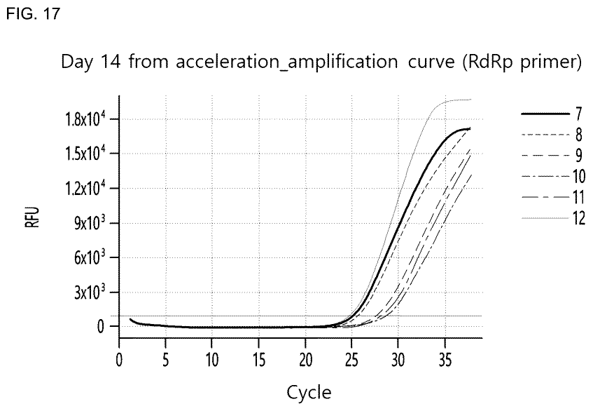

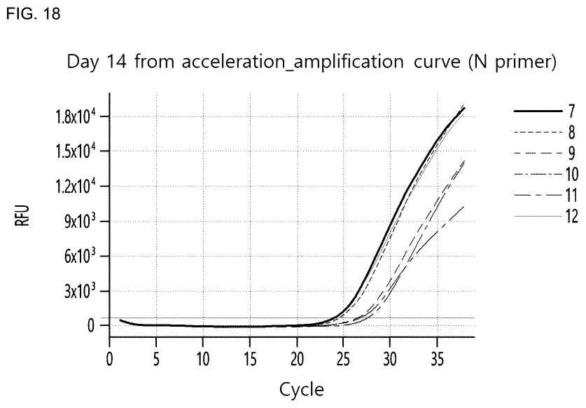

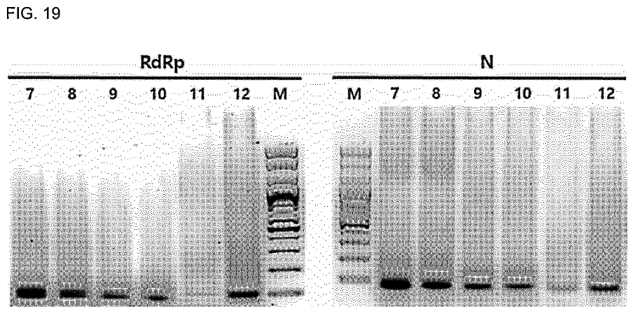

A nucleic acid amplification reaction-stabilizing composition was prepared as an example of an enzyme-stabilizing composition including the polymer of a HPC-BSA conjugate, synthesized according to Synthesis Example 2 instead of the polymer of a PUL-BSA conjugate, synthesized according to Synthesis Example 1, and a nucleic acid amplification reaction was performed using the same manner as in Experimental Examples 1 and 2, and whether the target nucleic acid was amplified or not was detected using electrophoresis. The analysis results are illustrated in FIG. 12. The activity of the polymerase was stably maintained in the composition to which the polymer in which BSA is conjugated to HPC as a polysaccharide was added, whereas the activity of the polymerase was remarkably reduced in the composition to which the simple mixture of HPC and BSA was added.

### Experimental Example 4: Comparison of Synthesis Examples 1 and 2 and Comparative Synthesis Example 1

It was evaluated whether the dextran-BSA conjugate of Comparative Synthesis Example 1, the pullulan-BSA conjugate of Synthesis Example 1, and the hydroxypropyl cellulose-BSA conjugate of Synthesis Example 2 stabilize an enzyme. Specifically, an enzyme-stabilizing composition was prepared in the same manner as in Example 1, a nucleic acid amplification reaction was performed in the same manner as in Experimental Examples 1 and 2, and whether the target nucleic acid was amplified or not was detected using electrophoresis. HPC refers to hydroxypropyl cellulose, and BSA refers to bovine serum albumin.

Components added to the nucleic acid amplification reaction-stabilizing composition and the contents thereof are shown in Table 2 below.

TABLE 2

| | Group | | | | | |
|---|---|---|---|---|---|---|
| | Pullulan- | HPC- | | | | |
| | BSA | BSA | Dextran-BSA | | Frozen | Freeze-drying |
| | (40° C.) | (40° C.) | (40° C.) | | (−20° C.) | (40° C.) |
| Classification | 7 | 8 | 9 | 10 | 11 | 12 |
| Polymer (5%) | 4 μl | 6 μl | 4 μl | 6 μl | — | — |
| Premix* | 4 μl | 4 μl | 4 μl | 4 μl | 4 μl | 4 μl |
| Additive* | 5 μl | 5 μl | 5 μl | 5 μl | — | — |
| TE buffer | 7 μl | 5 μl | 7 μl | 5 μl | — | 16 μl |
| Total volume | 20 μl | 20 μl | 20 μl | 20 μl | 4 μl | 20 μl |

Premix: Mix Taq polymerase (5 unit/μl) and 2.5 mM dNTP
Additives: Mannitol, PEG 10000, Trehalose, and Betaine (5 wt % to 40 wt %)

The results thereof are illustrated in FIGS. 13 to 19. FIGS. 13 to 18 illustrate the nucleic acid amplification reaction according to the period of acceleration, and FIG. 19 illustrates the electrophoresis results.

As illustrated in FIGS. 13 to 19, it can be seen that, compared with lanes 9 and 10 using Comparative Synthesis Example 1, lanes 7 and 8 using a conjugate of BSA and any one polysaccharide selected from pullulan and hydroxypropyl cellulose exhibited a remarkably improved enzyme-stabilizing effect.

While the present disclosure has been described on the basis of exemplary embodiments and examples of the present disclosure, the present disclosure is not limited to the technical ideas described in the embodiments and the examples. Rather, various modifications and changes may be easily made by those of ordinary skill in the art to which the present disclosure pertains, on the basis of the above-described embodiments and examples. However, it is apparent that these modifications and changes all fall within the scope of the present disclosure as defined in the appended claims.

1. A polymer in which serum albumin is conjugated to a polysaccharide.

2. The polymer of claim 1, wherein the polysaccharide is selected from the group consisting of cellulose, hydroxypropyl cellulose, hydroxypropyl methylcellulose, glycosaminoglycan, pullulan, alginic acid, carrageenan, aribinogalactan, hemicellulose, dextran, chitosan, glycol chitosan, starch, and a combination thereof.

3. A method of synthesizing a polymer in which serum albumin is conjugated to a polysaccharide, the method comprising:
dissolving a mixture of a polysaccharide and serum albumin, at a concentration of 0.1% (w/v) to 20% (w/v) in distilled water; and
inducing hydrothermal synthesis by causing a reaction in a solution in which the polysaccharide and the serum albumin are dissolved, at 40° C. to 200° C. for 6 hours to 72 hours.

4. The method of claim 3, further comprising, after the induction of the hydrothermal synthesis, performing dialysis treatment of the hydrothermally synthesized polymer.

5. The method of claim 4, wherein the dialysis treatment is performed in distilled water for 24 hours to 72 hours using a 100-500 molecular weight cut-off (MWCO) dialysis membrane.

6. The method of claim 4, further comprising, after the dialysis treatment, freezing the polymer at a temperature of −20° C. to −70° C., and then freeze-drying the frozen polymer.

7. An enzyme-stabilizing composition comprising a polymer in which serum albumin is conjugated to a polysaccharide.

8. The enzyme-stabilizing composition of claim 7, wherein the polysaccharide is selected from the group consisting of cellulose, hydroxypropyl cellulose, hydroxypropyl methylcellulose, glycosaminoglycan, pullulan, alginic acid, carrageenan, aribinogalactan, hemicellulose, dextran, chitosan, glycol chitosan, starch, and a combination thereof.

9. The enzyme-stabilizing composition of claim 7, further comprising a nucleic acid polymerase.

10. The enzyme-stabilizing composition of claim 9, further comprising at least one selected from a primer, a probe, a deoxyribonucleotide triphosphate (dNTP), and a nucleotide triphosphate (NTP).

11. The enzyme-stabilizing composition of claim 7, further comprising an additive for a nucleic acid amplification reaction.

12. A method of amplifying a nucleic acid molecule, the method comprising:
preparing the enzyme-stabilizing composition according to claim 7;
adding a biological sample comprising the nucleic acid molecule to the enzyme-stabilizing composition; and
performing a nucleic acid amplification reaction by using the enzyme-stabilizing composition to which the biological sample has been added.

13. The method of claim 12, wherein, before the biological sample is added to the enzyme-stabilizing composition, the enzyme-stabilizing composition is kept frozen at −20° C. to −70° C., and then freeze-dried.

14. The method of claim 12, wherein the nucleic acid amplification reaction is performed using a method selected from the group consisting of polymerase chain reaction (PCR), reverse transcription-polymerase chain reaction (RT-PCR), real-time PCR, reverse transcription, complementary DNA synthesis, loop-mediated isothermal amplification (LAMP), real-time nucleic acid sequence-based amplification (NASBA), strand displacement amplification (SDA), multiple displacement amplification (MDA), rolling circle amplification (RCA), ligase chain reaction (LCR), helicase dependent amplification (HDA), a ramification-extension amplification method (RAM), an in-vitro transcription-based amplification system (TAS), and a combination thereof.

15. A method of analyzing the presence of a target nucleic acid molecule in a biological sample, the method comprising:
preparing the enzyme-stabilizing composition according to claim 7;
adding a biological sample comprising the nucleic acid molecule to the enzyme-stabilizing composition;
performing a nucleic acid amplification reaction by using the enzyme-stabilizing composition to which the biological sample has been added; and
detecting whether the nucleic acid molecule is amplified.
